(19) United States
(12) Plant Patent Application Publication
(10) Pub. No.: US 2013/0167273 P1
(43) Pub. Date: Jun. 27, 2013

(54) Pear tree named "CELINA'

(50) ;

(71) Applicant: Graminor AS, Ridabu (NO)

(72) Inventor: Stein Harald HJELTNES, Leikanger (NO)

(73) Assignee: Graminor AS, Ridabu (NO)

(21) Appl. No.: 13/694,445

(22) Filed: Dec. 4, 2012

(30) Foreign Application Priority Data
Dec. 26, 2011 (QZ) 2011/3173

(51) Int. Cl. A01H 5/00

(52) U.S. Cl. PLT/176

(57) ABSTRACT

A new and distinct Pyrus communis pear tree variety named ‘CELINA’ particularly characterized by its green-yellow fruit with a red blush, pyriform/straight to convex fruit shape, fine fruit texture, medium fruit size, high yield, early fruit maturation, and resistance to scab.

### LATIN NAME OF THE GENUS AND SPECIES OF THE PLANT CLAIMED

Pyrus communis

### VARIETY DENOMINATION

‘CELINA’

### BACKGROUND OF THE INVENTION

The present invention relates to a new and distinct variety of pear tree, botanically known as Pyrus communis of the Rosaceae family, and hereinafter referred to by the variety denomination ‘CELINA’.

The new Pyrus variety is a product of a controlled breeding program conducted by the inventor, Stein Harald Hjeltnes, in [symbol] , Kristianstad, Sweden. The objective of the breeding program was to develop a new Pyrus variety with early maturation, high fruit quality, good shelf life, attractive fruits, high and stable production.

The new Pyrus variety originated from a cross made by the inventor in 1985 in [symbol] , Kristianstad, Sweden. The female or seed parent is the Pyrus communis variety designated ‘Colorée de Juillet’, unpatented. The male or pollen parent is the Pyrus communis variety designated ‘Williams’, unpatented. The new Pyrus variety was discovered and selected by the inventors within the progeny of the stated cross in a controlled environment in 1997 in Njøs, Leikanger, Norway.

### BRIEF DESCRIPTION OF THE INVENTION

The following traits have been repeatedly observed and are determined to be unique characteristics of ‘CELINA’ which in combination distinguish this pear tree as a new and distinct variety:

1. Fruit color: dark red blush with small red dots at lenticels, green-yellow background

2. Fruit shape: pyriform, straight to convex

3. Fruit flesh texture: fine

4. Fruit size: medium

5. Fruit number: high, productive

5. Time of maturation: early

6. Leaf color: dark green

7. Leaf shape: medium long and wide

8. Leaf texture: leathery

9. Leaf size: medium-large

10. Flower number: 10 flowers per clusters, flowers abundantly

11. Flower color: white with pinkish anthers

12. Flowering time: mid-season

13. Tree growth habit: very vigorous, spreading

14. Disease resistance: resistant to scab

In comparison to the parental varieties, ‘COLORÉE DE JUILLET’ and ‘WILLIAMS’, ‘CELINA’ differs primarily in the traits listed in Table 1.

TABLE 1
Comparison with Parent Varieties

| | | Female Parent | |
|---|---|---|---|
| | | ‘COLORÉE DE | Male Parent |
| | New Variety | JUILLET’ | ‘WILLIAMS’ |
| Trait | ‘CELINA’ | (unpatented) | (unpatented) |
| Over color | dark red, speckled, | brown to dark red, | Faint blush |
| | intense | dotted and striped | |
| Fruit shape | Pyriform, straight to | Ovate, convex | Oblong-obtuse- |
| | convex | | pyriform |
| Fruit texture | Fine | Fine | Fine |
| Fruit size | 150-180 grams | 100-120 grams | 100-150 grams |
| Time of | Early | Extremely early | Early |
| maturation | | | |

Of the many commercial varieties known to the present inventor(s), the most similar in comparison to the new Pyrus variety ‘CELINA’ is the Pyrus variety ‘Forelle’, (unpatented), in the following characteristics described in Table 2:

TABLE 2
Comparison with Similar Variety

| | | Comparison Variety |
|---|---|---|
| | New Variety | ‘FORELLE’ |
| Characteristic | ‘CELINA’ | (unpatented) |
| Over colour | intense dark red, speckled | Reddish with trout-like speckles |
| Fruit shape | Pyriform | Oblong-obovate-pyriform |
| Fruit texture | Fine | Fine |
| Fruit size | 150-180 grams | 150-180 grams |
| Time of | Early | Medium-late |
| maturation | | |

### BRIEF DESCRIPTION OF THE DRAWINGS

The accompanying photographs illustrate the overall appearance of the new Pyrus variety ‘CELINA’ showing the colors as true as is reasonably possible with colored reproductions of this type. Colors in the photographs may differ slightly from the color values cited in the detailed botanical description, which accurately describe the color of ‘CELINA’.

FIG. 1—shows a flower cluster of ‘Celina’

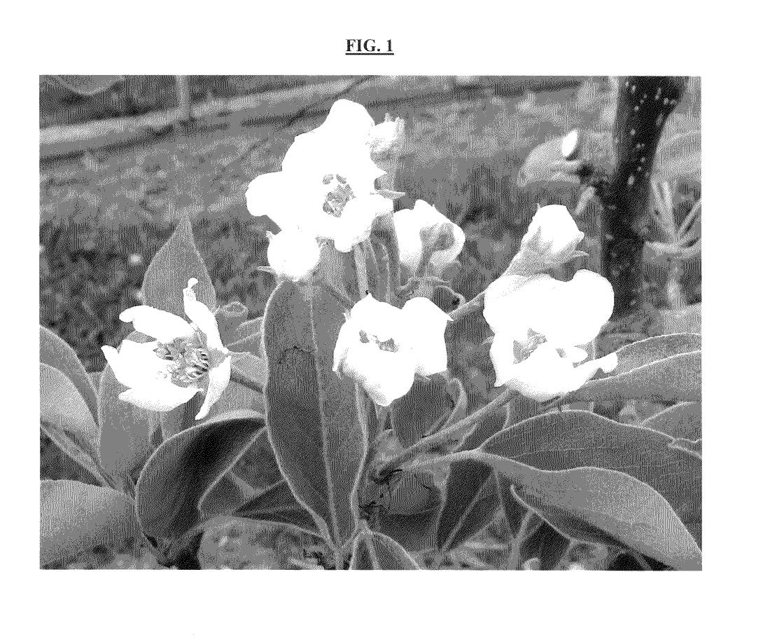

FIG. 2—shows typical fruits of ‘Celina’

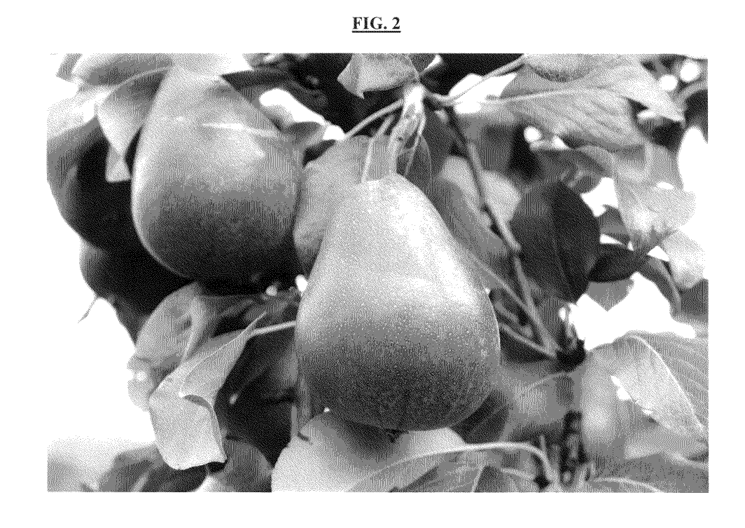

FIG. 3—shows well exposed ‘Celina’ fruits on a sunny day.

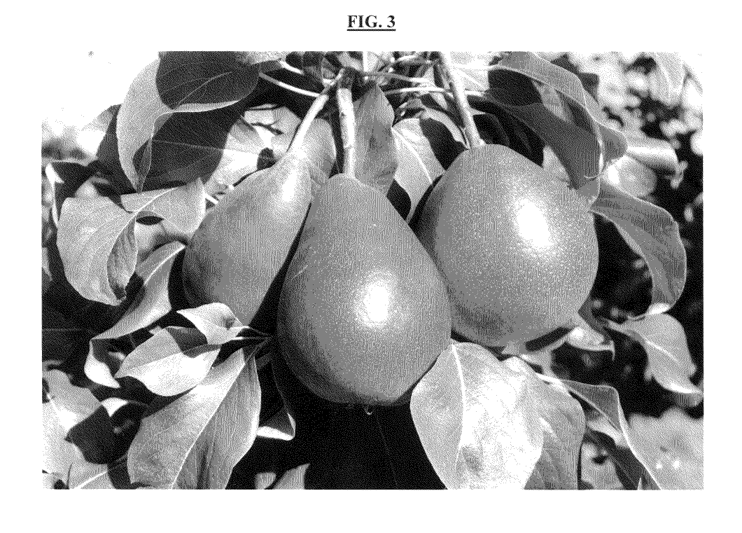

FIG. 4—shows 14 year old trees of ‘Celina’ with fruitlets on Aug. 1, 2012 (about 7 weeks prior to harvest).

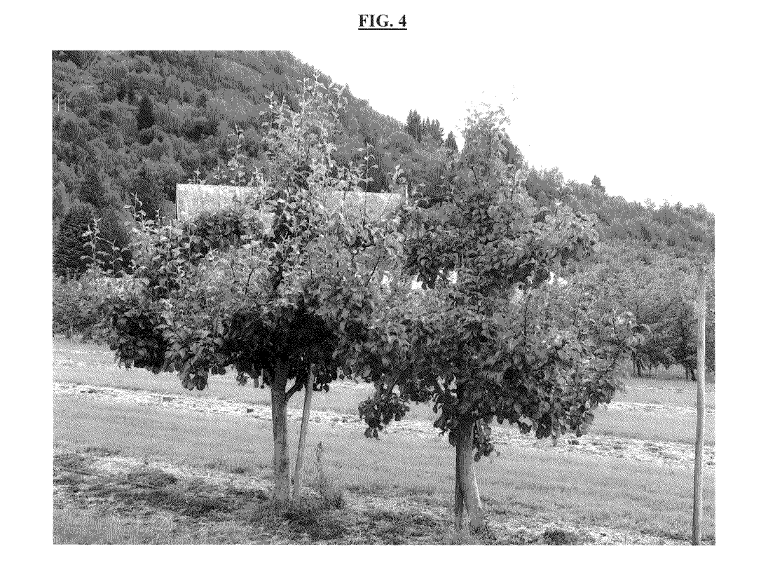

### DETAILED BOTANICAL DESCRIPTION

The new Pyrus variety ‘CELINA’ has not been observed under all possible environmental conditions. The phenotype of the new variety may vary with variations in environment such as temperature, light intensity, and day length without any change in the genotype of the pear tree.

The aforementioned photographs, together with the following observations, measurements and values describe trees of ‘CELINA’ as grown in Njøs, Leikanger, Norway, under conditions which closely approximate those generally used in commercial practice. Field trials were done with black plastic mulching and trickle irrigation, spaced 4×2 meter. Mean temperatures in May-September were approximately 13° C., with (rarely) maximum temperatures above 25° C. Daily mean temperatures were 15-20° C., 30-years normal average was 15° C. Winters were mild winters, rarely under −10° C. Mean temperature in the coldest month was −1° C. Fertilization was 50 kg N, 20 kg P and 70 kg K per ha).

Unless otherwise stated, the detailed botanical description includes observations, measurements and values based on 6 to 14 year old ‘CELINA’ trees grown in Njøs—Leikanger, Norway, from 1998 to 2012. Quantified measurements are expressed as an average of measurements taken from a number of trees of ‘CELINA’. The measurements of any individual tree, or any group of trees, of the new variety may vary from the stated average.

Color references are made to The Royal Horticultural Society Colour Chart (R.H.S.), fifth edition, 2007, except where general colors of ordinary significance are used. Color values were taken under daylight conditions at approximately 2 pm in Njøs—Leikanger, Norway.

All of the trees of ‘CELINA’, insofar as they have been observed, have been identical in all the characteristics described below.
Classification:
   Botanical.—Pyrus communis.
   Commercial.—Pyrus cv. Celina.
   Variety name.—CELINA.
Parentage:
Female or seed parent: Pyrus variety designated ‘COLORÉE DE JUILLET’ (unpatented)
Male or pollen parent: Pyrus variety designated ‘WILLIAMS’ (unpatented)
Propagation: Grafted on Pyrus communis seedling.
Growing conditions:
   Light intensities.—Full sunlight.
   Temperature.—During day, grown in range of 10° C. to 25° C., and during evening, grow in range of 5° C. to 15° C.
   Fertilization.—A balanced fertilizer with level of 50 kg N, 20 kg P and 70 kg K per ha.
   Growth regulators.—No growth regulators applied.
   Pruning or trimming requirements.—no specific requirements.
Tree:
Age: Observed trees were 14 years old.
Vigor: High vigor; early onset of production; high productivity and abundant early flowering.
Form: Spreading and pyramidal
Habit: A large sized tree with one trunk and usually one leader; main branches spreading; crown symmetrical and pyramidal in shape. Tree is trained to a defined system, that has been modified the last 4 years.
Branching habit: Main branches angle is 60° to 90° with respect to trunk if allowed to grow naturally. Trees have not been grown naturally.
Density: Medium density
Size at maturity: Depends on space available. Truncated to:
   Height.—About 3.5 m.
   Spread.—About 2 m.
Trunk:
   Height (up to leaders).—About 0.8 m.
   Diameter.—About 0.11 m.
   Texture.—At first, smooth with numerous lenticels, and the trunk stays smooth.
   Bark color.—Primarily RHS 195C, with RHS 162C underbark.
   Trunk lenticels.—Length: About 15 mm Width: About 2 mm Color: RHS 199B Density: 2/cm².
Branches:
   Number per tree.—About 8.
   Length.—Varies due to the shape of tree; maximum of 100 cm to 140 cm; minimum of 40 cm to 60 cm.
   Diameter.—About 30 mm to 50 mm.
   Surface texture.—smooth.
   Color.—Mature (after about 4 years): RHS 197B New Growth: RHS 183C.
   Internode length.—About 3.2 cm to 3.6 cm.
   Internode diameter.—About 6 mm to 7 mm.
   Branch lenticels.—Length: About 3 mm Width: About 1 mm Color: RHS 197B Density: 2/cm².
Spur:
   Present.—Yes.
   Distance between each spur.—about 20 mm to 40 mm (on the four year old branches).
   Diameter of each spur.—About 8.
   Number of fruit per spur.—About 1 to 3.
Leaves:
Arrangement: Alternate
Lamina:
   Size.—Length: About 100 mm (from 95 to 105 at fully expanded leaf) Width: About 65 mm (from 62 to 68 at fully expanded leaf) Length/width ratio: 1.5.
   Overall shape.—curved, not regular.
   Base shape.—Acute to right angled.
   Apex shape.—acute.
   Margin.—crenate, undulated.
   Texture.—Upper surface: glabrous Under surface: glabrous.
   Pubescence.—Upper surface: some silver-colored at the main vein, else glabrous Under surface: some silver-colored at the main vein, some at secondary veins Color (mature leaves): Upper surface: RHS 135A Under surface: RHS 136B.
   Color (immature leaves).—Upper surface: RHS 144B Under surface: RHS 144A.
Venation:
   Type.—venation from central vein to the leaf edge.
   Color.—RHS 145B.
Petiole:
   Length.—About 50 mm.
   Diameter.—About 1 mm.
   Color.—RHS 145B.
Stipule:
   Arrangement.—adnate.
   Length (distance of stipules from basal attachment of petiole).—About 4 mm to 5 mm.
   Width.—About 0.5 mm to 1 mm.
Fruit:
Keeping quality: The fruit keeps well on the tree and doesn't easily drop. It can be stored in cold temperature conditions for up to 3 months without losing firmness and juiciness. It has a shelf life of at least 1 week without losing firmness and juiciness. Firmness is approximately at 1.8 kg/cm² after 12 days at 20° C. following 2 months of storage at −0.5° C. More than 3 months of storage in standard atmosphere will give faster drop in firmness. 1.4 kg/cm will be experienced in less than one week. This behavior is typical when harvesting at about 6 kg/cm². weeks.
Maturity when described: Ripe for eating
Maturity period after full bloom: About 118 days after full bloom on May 20^{th} in Norway
Type: Pome
General shape: Turbinate
Average weight: About 150 g
Fruit size:
   Average height.—About 75 mm.
   Average diameter (at widest point).—About 65 mm.
   Height/thickness ratio.—1.15.
Stem:
   Length.—About 25 mm.
   Diameter.—About 3.5 mm.
   Color.—RHS B144.
Basin:
   Depth.—About 1 mm.
   Width.—About 15 mm.
Calyx:
   Depth.—About 1 mm.
   Width.—About 25 mm.
   Crowning at calyx end.—semi-closed.
   Calyx tube.—Funnel shaped.
Skin:
   Thickness.—Medium to thin.
   Texture.—fine.
   Firmness (at picking time).—5 to 6 kg/cm².
   Color.—RHS 144C.
   Lenticels.—Length: About 1 mm Width: About 1 mm Color: RHS 144A Density: 30 n°/cm².
Flesh:
   Color.—RHS 1D.
   Texture.—tender, fine, juicy.
   Aroma.—slight.
   Sugar content (at picking time).—11 to 13 Brix.
   Acidity/starch (at picking time).—1.3 to 2.0 g/lt.
   Core.—Symmetry of core: symmetric Distinctness of core lines: green, medium visible Locules: Number (per fruit): 5 Length: About 16 mm Width: About 3 mm Form: closed.
Seeds:
   Number per fruit.—About 2 to 10.
   Number per locule.—About 2.
   Shape.—oval.
   Length.—About 10 mm.
   Width.—About 4 mm.
   Color.—RHS 202A.
Use: Fresh market.
Disease/pest resistance: Resistance to scab.
Disease/pest susceptibility: None observed.
Winter hardiness: Tolerant to temperatures down to −20° C. without observed damage to wood and buds of dormant pear trees (observed in experimental field in Eastern Norway); but open flowers and young fruitlets are killed by exposure to −3° C., depending on the length of exposure.
Drought/heat tolerance: No information available

I claim:

1. A new and distinct Pyrus communis pear tree variety named ‘CELINA’, as illustrated and described herein.
